(19) United States
(12) Patent Application Publication
(10) Pub. No.: US 2023/0105122 A1
(43) Pub. Date: Apr. 6, 2023

(54) Error corrected method mitigates systematic error via sequencing DNA data of the surrounding flow cells of the variants on Patterned Flow Cell

(71) Applicant: Shou-Chien KUO, Redwood City, CA (US)

(72) Inventor: Shou-Chien KUO, Redwood City, CA (US)

(21) Appl. No.: 17/959,615

(22) Filed: Oct. 4, 2022

Related U.S. Application Data
(60) Provisional application No. 63/252,275, filed on Oct. 5, 2021.

(51) Int. Cl. C12Q 1/6869

(52) U.S. Cl. CPC C12Q 1/6869; C12Q 2600/166; C12Q 1/6855

(57) ABSTRACT

A method of determining a target nucleic acid of interest using synthetic Phix sequences designed to match the target nucleic acid fragments. The sequencing error profile was generated using the synthetic Phix L and synthetic Phix S, and the sequencing read locations information on the patterned flow cell.

### CROSS-REFERENCE TO RELATED APPLICATIONS

This application claims priority of U.S. Provisional Application No. 63/252,275 filed on Oct. 5, 2021 under 35 U.S.C. § 119(e), the entire contents of all of which are hereby incorporated by reference.

### BACKGROUND OF THE INVENTION

Next-generation sequencing platforms, Illumina's sequencers or others, employ fluorescently labeled reversible terminators integrated into the complementary strand of the DNA template. At the same time as the synthesis of the nuclide process, the exciting single color for each base is captured by a sensitive CCD/CMOS camera. These captured images are processed into signals used to infer the sequence of nucleotides, also known as base recognition. The accuracy of base recognition is measured by the Q score (Phred quality score), a standard index for evaluating the accuracy of sequence execution. The Q score is defined as a logarithmic correlation with the probability of a base call error.

The Illumina sequencing platforms carry several biases, including phasing (lagging), pre-phasing(leading), signal decay, and cross-talk [1]. Next-generation sequencing is used in many clinical applications that demand high accuracy and quality sequencing data. However, high sequencing accuracy must reduce errors or be aware of errors from DNA library preparation, amplification, and sequencing. In addition, in early-stage tumor patients, the mutation rate[2] may be below 1%, which is close to the error rate.

PhiX has been used as a control for Illumina sequencing runs. Illumina recommends using PhiX in library preparations for low diversity samples and quality control[3].

Patterned flow cells are used in the next generation sequencer and have structured wells across patterned flow cell surfaces at fixed locations. The Patterned flow cell design increases the density of the reaction wells and the sequencing throughput.

### SUMMARY OF THE INVENTION

The present invention provides the method of profiling the sequencing error rate using synthetic Phix for specific sequences of nucleic acid fragments of interest, starting from library preparation to sequencing outcome and sequencing to sequencing outcome. The synthetic Phix types include synthetic Phix L for profiling the error rate starting from library preparation and synthetic Phix S for profiling the error rate starting from sequencing.

The invention provides a method of profiling the sequencing error rate on the known well/reaction locations on the patterned flow cell.

In certain aspects of the present invention, the method comprises the steps of (a) obtaining DNA molecules of sample, (b) designing the synthetic Phix L and synthetic Phix S for the interest target sequences, (c) amplifying individual DNA molecules, and the synthetic Phix L, (d) sequencing the individual DNA molecules, the synthetic Phix L and the synthetic Phix S in the same run, (e) generating error profiles using the synthetic Phix L and the synthetic Phix S within the surrounding the target wells on the patterned flow cell, and (f) analyzing the target mutation and comparing with error profiles.

### BRIEF DESCRIPTION OF DRAWINGS

FIG. 1a shows the synthetic Phix L with no binding flow cell oligonucleotide adapters, and FIG. 1b shows the synthetic Phix S with binding the flow cell oligonucleotides adapters.

FIG. 2 shows the synthetic Phix L, a PCR amplification product, ligates binding the flow cell oligonucleotide adapters.

FIG. 3a illustrates the top view of the patterned flow cell, and FIG. 3b illustrates the clusters of DNA, the synthetic Phix L, and the synthetic Phix S.

### DETAILED DESCRIPTION OF THE INVENTION

The disclosure provides a method for profiling sequencing errors rate and applying it to enhance the quality of the mutation call on the next generation sequencer patterned flow cell.

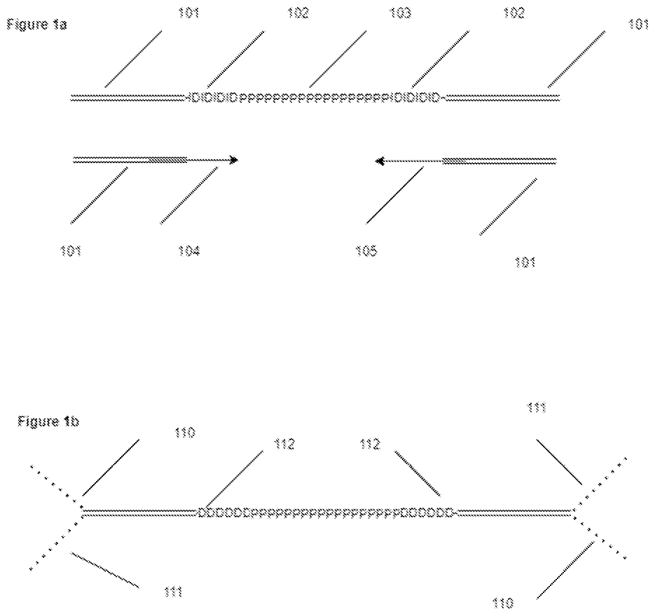

In one embodiment, the method for preparing a synthetic Phix L includes the following steps: (a) selecting a DNA target fragment of interest, for example, a length of 15 bases polyA sequence AAAAAAAAAAAAAAA. (b). designing the PCR amplifiable synthetic Phix L containing AAAAAAAAAAAAAAA sequence. FIG. 1a illustrates an exemplary synthetic Phix L sequence. 101 is an adapter sequence for ligating the flow cell adapters that ligate the oligonucleotide on the flow cell. 102 is the identifier sequence, IDIDIDID, of PPPPPPPPPPPPPP, for example, AAAAAAAAAAAAAAA sequence. 103 is the target sequence, PPPPPPPPPPPPPPPPPPPPPP, for example, AAAAAAAAAAAAAAA. (c). designing the PCR amplification primers 104 and 105 of the synthetic Phix L.

In one embodiment, the method for preparing a synthetic Phix S includes the following steps: (a) selecting a DNA target fragment of interest, for example, a length of 15 bases polyA sequence AAAAAAAAAAAAAAA. (b). designing the synthetic Phix S, ready to be sequenced, containing AAAAAAAAAAAAAAA sequence and adapter for ligating the flow cell adapters that ligate the oligonucleotide on the flow cell. FIG. 1b illustrates an exemplary synthetic Phix S sequence. 110 is an adapter sequence for ligating the oligonucleotide on the flow cell. 112 is the identifier sequence, DDDDDD, of PPPPPPPPPPPPPP, for example, AAAAAAAAAAAAAAA sequence.

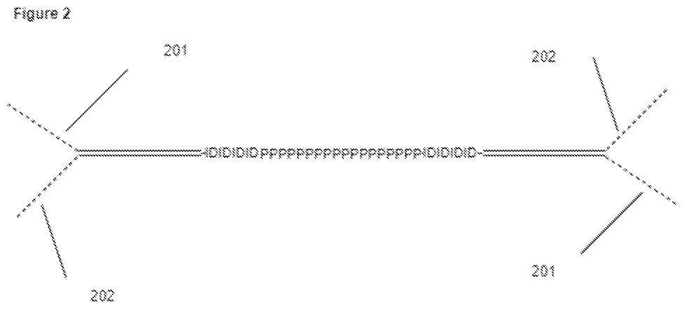

In one embodiment, the methods of applying flow cell binding adapters to both ends of double-stranded DNA fragments include the double-stranded DNA molecules, and the double-stranded synthetic Phix L. FIG. 2 illustrates an exemplary of the amplification product of double-stranded synthetic Phix L-adapter. 201 and 202 are the adapters for ligating the oligonucleotide on the flow cell.

In one embodiment, the method for amplifying the target region of interest includes the double-stranded target DNA-adapter molecules and the double-stranded synthetic Phix L-adapter to obtain a plurality of amplified polynucleotides.

In one embodiment, the method for performing sequencing on the sequences of the target region of interest includes the double-stranded target DNA-adapter molecules, the double-stranded synthetic Phix L-adapter, and the double-stranded synthetic Phix S library.

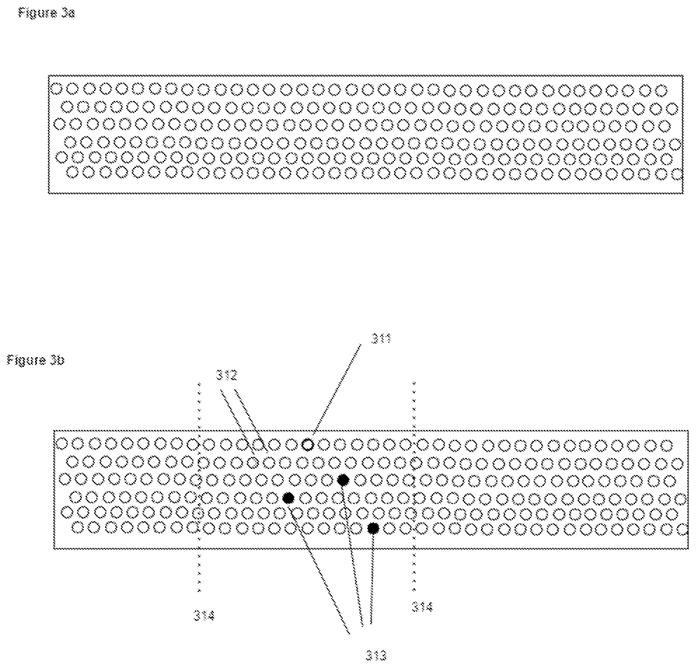

In one embodiment, the method for performing the sequence data analysis and modeling includes the variant calling of the DNA molecules, the error profile of the synthetic Phix L, the error profile of the synthetic Phix S, and the synthetic Phix L and the synthetic Phix S wells information on the flow cell surrounding the DNA molecules. FIG. 3a illustrates an exemplary patterned flow cell and the oligonucleotide reaction area of the patterned flow cell. FIG. 3b illustrates an exemplary of the DNA molecule cluster 311, the synthetic Phix L cluster 312, and the synthetic Phix S cluster 313 on the patterned flow cell. 314s are the cluster region edges surrounding the DNA molecule.

In some embodiments, the point mutation rate can be calculated using (total mutation base)/(total mutation base+total non-mutation base) at a locus of the reference genome. The error rate of the synthetic Phix L or the error rate of the synthetic Phix S can be calculated using (total mismatch base)/(total mismatch base+total match base) at the same locus of the point mutation base of the reference genome. If the point mutation rate is lower than the error rate of the synthetic Phix L or the synthetic Phix S. The point mutation may not be considered an actual mutation.

In some embodiments, the sequencing reads are generated with a sequencer that employs the following:
a) sample collection,
b) sample and the synthetic Phix L processing preliminary to sequencing,
c) sequencing the sample and the synthetic Phix L library and the synthetic Phix s, and
d) analyzing sequence data and deriving mutation calls.

1. A method for profiling the sequencing error happening in the library preparation or/and sequencing of a DNA sequence, the method comprising:
(a). adding the synthetic target sequence pattern Phix L fragments to the DNA library;
(b). amplifying the DNA molecules and the synthetic Phix L;
(c). adding the ready-to-sequence synthetic Phix S fragments to the amplified DNA molecules and the amplified synthetic Phix L;
(d). sequencing the ready-to-sequence synthetic Phix S fragments, the amplified DNA molecules and the amplified synthetic Phix L; and
(e). analyzing the target sequence DNA read and the error profile of the two different types of synthetic Phix L and synthetic S in the surrounding wells of the target sequence DNA read on the patterned flow cells.

2. The method of claim 1, where the step of adding the synthetic target sequence pattern Phix L fragments includes the designed target interest sequence pattern synthetic Phix L and PCR amplification primers for the designed target interest sequence pattern synthetic Phix L.

3. The method of claim 1, where the step of adding the ready-to-sequence synthetic Phix S fragments includes the designed target interest sequence pattern synthetic Phix S with flow cell required adapters ready for sequencing.

4. The method of claim 1, where the step of analyzing the target sequence DNA includes using the error profile generated from the sequencing results of the two types synthetic Phix L and Phix S around the surrounding of the DNA sequence cluster location.
